(19) United States
(12) Patent Application Publication
(10) Pub. No.: US 2024/0230542 A1
(43) Pub. Date: Jul. 11, 2024

(54) METHOD FOR HIGHLY-SENSITIVE AND RAPID DETECTION OF PESTICIDE RESIDUE BASED ON IMPRINTED METAL-ORGANIC FRAMEWORK PROBE

(71) Applicant: JIANGSU UNIVERSITY, Zhenjiang (CN)

(72) Inventors: Xinai ZHANG, Zhenjiang (CN); Xueyue HUANG, Zhenjiang (CN); Jiyong SHI, Zhenjiang (CN); Xiaobo ZOU, Zhenjiang (CN); Xiaowei HUANG, Zhenjiang (CN); Zhihua LI, Zhenjiang (CN); Zhiming GUO, Zhenjiang (CN); Di ZHANG, Zhenjiang (CN); Xiaodong ZHAI, Zhenjiang (CN); Xuetao HU, Zhenjiang (CN); Tingting SHEN, Zhenjiang (CN)

(73) Assignee: JIANGSU UNIVERSITY, Zhenjiang (CN)

(21) Appl. No.: 18/008,687

(22) PCT Filed: Dec. 22, 2021
(86) PCT No.: PCT/CN2021/140485

§ 371 Date: Dec. 7, 2022

(30) Foreign Application Priority Data
Dec. 8, 2021 (CN) 202111494602.3

(51) Int. Cl. G01N 21/78; G01N 21/77

(52) U.S. Cl. CPC G01N 21/78; G01N 2021/7759

(57) ABSTRACT

A method for highly-sensitive and rapid detection of a pesticide residue based on an imprinted metal-organic framework (MOF) probe is provided. A molecularly imprinted MOF enzyme-mimic probe is used as a colorimetric probe to catalyze the oxidation of a substrate, thereby enabling a color change of a system; a low-cost filter paper is used as a substrate for supporting the colorimetric probe, including a quality control zone, a standard zone, and a detection zone; in the quality control zone, the optimal colorimetric analysis parameters can be selected according to the temperature, humidity, and light, etc. of an environment to be tested; the standard zone is a standard colorimetric zone obtained through the dropwise addition of standards with different concentrations and is provided to establish a colorimetric analysis mathematical model; and the detection zone is provided for the detection of an actual sample.

### CROSS REFERENCE TO THE RELATED APPLICATIONS

This application is the national phase entry of International Application No. PCT/CN2021/140485, filed on Dec. 22, 2021, which is based upon and claims priority to Chinese Patent Application No. 202111494602.3, filed on Dec. 8, 2021, the entire contents of which are incorporated herein by reference.

### TECHNICAL FIELD

The present disclosure belongs to the technical field of safety detection for agricultural products, and in particular relates to a method for highly-sensitive and rapid detection of a pesticide residue based on an imprinted metal-organic framework (MOF) probe.

### BACKGROUND

The extensive use of pesticides can effectively control the infestation of pests and diseases during the growth of crops. However, due to difficult degradation, strong toxicity, easy residues, and other disadvantages, pesticides are easy to accumulate in agricultural products, soil, and water sources, causing a great threat to the ecological environment and human health. Therefore, the detection of pesticide residues is very important. The current methods for detecting pesticide residues mainly include traditional methods such as high-performance liquid chromatography (HPLC) and liquid chromatography-mass spectrometry (LC-MS) and rapid detection methods such as electrochemical method, Raman method, and fluorescence method emerging in recent years. However, these methods have high detection sensitivity and high result accuracy, usually need to be conducted under laboratory conditions, involve a large reagent consumption, rely on special equipment, and require operators to have a high technical level, which makes it difficult to meet the needs of on-site and rapid detection of a pesticide residue. Therefore, it is particularly important to develop a portable method for in-situ monitoring and rapid field analysis.

Recently, the existing colorimetric probe detection methods for pesticide residues have the following deficiencies: 1. The existing finished test strips do not allow the timely adjustment of analysis parameters according to implementation conditions. Because the colorimetric analysis method is easily affected by surrounding environment factors such as temperature, humidity, and light, a test strip may have inaccurate signal output. 2. When a sample to be tested has a too-high or too-low concentration, the existing test strips are prone to large errors. 3. A color of an actual sample itself will interfere with a colorimetric signal to varying degrees. Without the use of a special instrument, it is impossible to achieve the accurate quantitative analysis of a target by determining a color change only with naked eyes and a colorimetric card. Due to the above factors, the colorimetric analysis methods are limited to qualitative and semi-quantitative detection, and can hardly be used for low-cost, high-sensitivity, and anti-interference detection of a target in a complex matrix sample.

In summary, it is a key and difficult task to develop a colorimetric analysis method that can be adapted to local conditions to achieve the sensitive and accurate analysis of a pesticide residue in a complex matrix sample without the aid of a special instrument.

### SUMMARY

In view of the deficiencies in the prior art, the present disclosure constructs a colorimetric test strip for highly-sensitive and rapid detection of a pesticide residue based on an imprinted MOF enzyme-mimic probe. A molecularly imprinted MOF enzyme-mimic probe is used as a colorimetric probe to catalyze the oxidation of a substrate, thereby enabling a color change of a system; and a low-cost filter paper is used as a substrate for supporting the colorimetric probe, including a zone A, a zone B, and a zone C, where the zone A is a quality control zone, the zone B is a standard zone, and the zone C is a detection zone. In the quality control zone, optimal colorimetric analysis parameters can be selected according to the temperature, humidity, and light, etc. of an environment to be tested; the standard zone is a standard colorimetric zone obtained through the dropwise addition of standards with different concentrations under the analysis conditions optimized in the quality control zone and is provided to establish a colorimetric analysis mathematical model; and the detection zone is provided for the detection of an actual sample. It should be noted that, before an actual sample is detected, a color change of the detection zone is observed in contrast to the standard zone, a concentration range of a pesticide residue in a test sample is preliminarily determined, and then a sample with a too-high or low-low pesticide residue concentration is adjusted to meet the requirements of accurate analysis; and then a smartphone is used to stably capture a color signal (RGB value) of a reaction system, and a colorimetric analysis mathematical model is established by calculating a Gray value to eliminate the interference of a color of the test sample itself on an end point color, thereby achieving the sensitive, accurate, real-time, and quantitative analysis of trace pesticide residues in multiple complex matrix samples.

In order to achieve the above objective of the present disclosure, the present disclosure adopts the following technical solutions.

The present disclosure provides a colorimetric test strip for high-sensitive and rapid detection of a pesticide residue based on an imprinted MOF probe. The colorimetric test strip is constructed by directly adding the imprinted MOF probe dropwise, and is divided into a quality control zone, a standard zone, and a detection zone. A zone A is the quality control zone configured to optimize the colorimetric analysis parameters in an on-site test environment; a zone B is the standard zone configured to detect a standard, where an RGB value is acquired through a camera function of a smartphone and a Gray value is calculated to establish a colorimetric analysis mathematical model; and a zone C is the detection zone, where a color change of the detection zone is observed in contrast to the standard zone, a concentration range of a pesticide residue in a test sample is preliminarily determined, and then a too-high or low-low pesticide residue concentration is adjusted to meet the requirements of accurate detection. The Gray value of the test sample is substituted into the colorimetric analysis mathematical model of the standard zone, thereby achieving the sensitive, accurate, and quantitative detection of a pesticide residue in a complex matrix sample.

The method specifically includes the following steps:
step 1: preparation of a colorimetric test paper, mainly including: synthesis of an imprinted MOF enzyme-mimic probe, preparation of a blank filter paper, and construction of a colorimetric sensing interface:
step 1.1: synthesis of an imprinted MOF enzyme-mimic probe: dissolving an MOF and aminopropyltriethoxysilane (APTES) in ammonia water to obtain a mixed solution; selecting a pesticide standard and denoting the pesticide standard as NY; adding the pesticide standard to the mixed solution, subjecting a resulting mixture to a first stirring, adding tetraethylorthosilicate (TEOS), and subjecting a resulting mixture to a second stirring; and centrifuging, washing, and drying to obtain the imprinted MOF enzyme-mimic probe;
step 1.2: preparation of a blank filter paper:
taking an ordinary filter paper, and dividing the ordinary filter paper into a zone A, a zone B, and a zone C, where the zone A is a quality control zone, the zone B is a standard zone, and the zone C is a detection zone;
step 1.3: construction of a colorimetric sensing interface:
dividing the quality control zone into a quality control subzone 1 and a quality control subzone 2; dividing the quality control subzone 1 into n zones from left to right that are denoted as H₁, H₂, H₃ . . . Hₙ₋₁, and Hₙ, respectively; and dividing the quality control subzone 2 into m zones from left to right that are denoted as I₁, I₂, I₃ . . . Iₘ₋₁, and Iₘ, respectively (where n and m are each an integer greater than 1);
step 2.1: establishment of the quality control zone:
step 2.1.1: determination of an optimal concentration of an imprinted MOF enzyme-mimic probe solution:
adding the imprinted MOF enzyme-mimic probe prepared in the step 1.1 to ethanol to obtain imprinted MOF enzyme-mimic probe solutions with different concentrations that are denoted as 1, 2 . . . n−1, and n, respectively, adding the imprinted MOF enzyme-mimic probe solutions 1, 2 . . . n−1, and n in a volume V1 dropwise to zones H₁, H₂, H₃ . . . Hₙ₋₁, and Hₙ of the quality control subzone 1, respectively, and allowing the zones to be dried; dissolving the NY in the step 1.1 into water to obtain an NY solution; adding the NY solution in a volume V2 dropwise to each of H₁, H₂, H₃ . . . Hₙ₋₁, and Hₙ of the quality control subzone 1, and allowing a reaction to occur for a period of time; adding a chromogenic reagent (where the chromogenic reagent includes 3,3′,5,5′-tetramethylbenzidine (TMB), hydrogen peroxide (H₂O₂), and NaAc-HAC with a pH of 4.0) in a volume V3 dropwise to each of H₁, H₂, H₃ . . . Hₙ₋₁, and Hₙ of the quality control subzone 1; observing color changes of H₁, H₂, H₃ . . . Hₙ₋₁, and Hₙ of the quality control subzone 1, acquiring an image and a corresponding RGB value of each zone, and further calculating a gray value; and determining a concentration of an imprinted MOF enzyme-mimic probe solution corresponding to a zone with the largest gray value as the optimal concentration of the imprinted MOF enzyme-mimic probe solution;
step 2.1.2: determination of an optimal concentration of a chromogenic reagent:
after the determination of the optimal concentration of the imprinted MOF enzyme-mimic probe solution in the step 2.1.1, adding an imprinted MOF enzyme-mimic probe solution with the optimal concentration in a volume V4 dropwise to zones I₁, I₂, I₃ . . . Iₘ₋₁, and Iₘ of the quality control subzone 2, and allowing the zones to be dried; adding the NY solution in the step 2.1.1 in a volume V5 dropwise to each of I₁, I₂, I₃ . . . Iₘ₋₁, and Iₘ of the quality control subzone 2, and allowing a reaction to occur for a period of time; adding a chromogenic reagent (where the chromogenic reagent includes TMB, hydrogen peroxide, and NaAc-HAC with a pH of 4.0) with different concentrations in a volume V6 dropwise to I₁, I₂, I₃ . . . Iₘ₋₁, and Iₘ of the quality control subzone 2, respectively; observing color changes of I₁, I₂, I₃ . . . Iₘ₋₁, and Iₘ of the quality control subzone 2, acquiring an image and a corresponding RGB value of each zone, and further calculating a gray value; and determining a chromogenic reagent concentration corresponding to a zone with the largest gray value as the optimal chromogenic reagent concentration;
step 2.2: establishment of the standard zone:
dividing the standard zone into n zones from top to bottom that are denoted as E₁, E₂, E₃ . . . Eₙ₋₁, and Eₙ, respectively; after the determination of the optimal concentration of the imprinted MOF enzyme-mimic probe solution in the step 2.1.1, adding the imprinted MOF enzyme-mimic probe solution with the optimal concentration in a volume V7 dropwise to a surface of each of E₁, E₂, E₃ . . . Eₙ₋₁, and Eₙ of the standard zone, and allowing the surfaces to be dried; preparing NY solutions with different concentrations, and denoting the NY solutions with different concentrations as C₁, C₂ . . . Cₙ₋₁, and Cₙ; adding the NY solutions with different concentrations in a volume V8 dropwise to E₁, E₂, E₃ . . . Eₙ₋₁, and Eₙ of the standard zone, respectively, and allowing a first reaction; and with the optimal chromogenic reagent concentration determined in the step 2.1.2, adding the chromogenic reagent in a volume V9 to each of E₁, E₂, E₃ . . . Eₙ₋₁, and Eₙ of the standard zone, and allowing a second reaction, so as to establish a standard colorimetric card for the standard zone, where a color of the standard colorimetric card for the standard zone remains unchanged for 20 min or more, which reserves enough time for the subsequent preliminary determination of a concentration of a pesticide residue in the detection zone;
step 2.3: acquiring chromogenic images of NY solutions with different concentrations corresponding to the standard colorimetric card for the standard zone in the step 2.2, and analyzing RGB values of the NY solutions with different concentrations; calculating corresponding Gray values according to equation (1), and denoting the Gray values as G₁, G₂, G₃ . . . Gₙ₋₁, and Gₙ, respectively,

$$Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}} \qquad ( 1 )$$

where R refers to a red value extracted from an image, G refers to a green value extracted from an image, B refers to a blue value extracted from an image, and Gray refers to a gray value;
establishing a colorimetric analysis mathematical model as follows based on Gray values of standard zones calculated by equation (1) and corresponding NY solution concentrations m: Y=k*m+b, where b and k are each a constant, and m is an NY solution concentration (μM); and denoting a discriminant value for an NY solution in the standard zone as P with a value range of M*(1-10%) to M*(1+10%), where M is a median for gray values corresponding to different NY solution concentrations in the standard zone;
step 2.4: establishment of the detection zone:
equally dividing the detection zone into N zones from left to right that are denoted as a sample zone 1, a sample zone 2, a sample zone 3 . . . a sample zone n−1, and a sample zone n; dividing the sample zone 1 into 1₁, 1₂, 1₃ . . . 1ᵢ, dividing the sample zone 2 into 2₁, 2₂, 2₃ . . . 2ᵢ, dividing the sample zone 3 into 3₁, 3₂, 3₃ . . . 3ᵢ, . . . dividing the sample zone n−1 into n−1₁, n−1₂, n−1₃ . . . n−1ᵢ, and dividing the sample zone n into n₁, n₂, n₃ . . . nᵢ;
pretreating n test samples to obtain test sample solutions that are numbered as a test sample solution 1, a test sample solution 2 . . . a test sample solution N, respectively; adding the imprinted MOF enzyme-mimic probe solution with the optimal concentration obtained according to the step 2.1.1 in a volume V10 dropwise to each of the N zones of the detection zone, and allowing the zones to be dried; and adding the test sample solution 1 in a volume V11 dropwise to each of 1₁, 1₂, 1₃ . . . 1ᵢ, adding the test sample solution 2 in a volume V11 dropwise to each of 2₁, 2₂, 2₃ . . . 2ᵢ . . . adding the test sample solution n−1 in a volume V11 dropwise to each of n−1₁, n−1₂, n−1₃ . . . n−1ᵢ, and adding the test sample solution n in a volume V11 dropwise to each of n₁, n₂, n₃ . . . nᵢ (where i and n are each an integer greater than 1), and allowing a reaction to occur for a period of time;
step 2.5: adding the chromogenic reagent with the optimal concentration obtained in the step 2.1.2 in a volume V12 dropwise to the N zones of the detection zone obtained in the step 2.4, and allowing a reaction to occur for a period of time; comparing colors of the N zones of the detection zone with colors of the standard colorimetric card for the standard zone established in the step 2.2, and preliminarily determining a concentration range of the pesticide residue in the test sample; and calculating a Gray value of the pesticide residue in the test sample according to equation (1), and when the Gray value is not in the value range of the discriminant value P, adjusting the concentration of the pesticide residue in the test sample,
where if the Gray value of the concentration of the pesticide residue in the test sample is greater than M*(1+10%), the test sample needs to be diluted until the Gray value is within the value range of the discriminant value P, and a dilution ratio is recorded; and
if the Gray value of the concentration of the pesticide residue in the test sample is smaller than the median M*(1-10%), the test sample needs to be concentrated until the Gray value is within the value range of the discriminant value P, and a concentration ratio is recorded;
after adjustment of the concentration of the pesticide residue, repeating the steps 2.4 and 2.5, calculating a Gray value G₀ of a chromogenic image according to equation (1), and comparing the Gray value with the discriminant value P; and if the G₀ value is within the value range of the discriminant value P, calculating a content of the pesticide residue in the test sample according to the colorimetric analysis mathematical model: Y₀=(G₀−b)/k, where M is a median for gray values corresponding to different NY solution concentrations in the standard zone, b and k are each a constant, and Y₀ is an adjusted pesticide concentration in the test sample.

Further, in the step 1.1, the MOF, the APTES, the ammonia water, the pesticide standard, and the TEOS are used in a ratio of (400-700) mg:(10-30) μL:(2-10) mL:(10-20) mg:(5-15) mL; the ammonia water has a volume fraction of 5% to 15%; and the first stirring and the second stirring are each performed for 5 min to 15 min.

Further, in the step 1.1, the pesticide standard includes an insecticide, a miticide, a bactericide, and an herbicide, and is specifically any one selected from the group consisting of thiacloprid, omethoate, abamectin, pyridaben, folpet, captan, alachlor, and atrazine.

Further, in the step 1.2, the zone A, the zone B, and the zone C are in an area ratio of 2:1:2.

Further, in the step 2.1.1, the NY solution has a concentration of 2 μM, and NY is any one selected from the group consisting of thiacloprid, omethoate, abamectin, pyridaben, folpet, captan, alachlor, and atrazine; the reaction is carried out for 5 min to 10 min; the imprinted MOF enzyme-mimic probe solution has a concentration of 1 mg/mL to 3 mg/mL; the chromogenic reagent is a mixed solution of TMB, H₂O₂, and NaAc-HAC with a pH of 4.0; and in the chromogenic reagent, the TMB, the H₂O₂, and the NaAc-HAC with a pH of 4.0 are mixed in a ratio of (0.4 mL-0.8 mL):(0.4 mL-0.8 mL):(0.1 mL-0.8 mL), and a concentration ratio of the TMB to the H₂O₂ is 1:20 to 5:1.

Further, in the step 2.1.1, the volume V1, the volume V2, and the volume V3 are in a ratio of 1:1:1, and are each 10 μL to 20 μL.

Further, in the step 2.1.2, the imprinted MOF enzyme-mimic probe solution has a concentration of 1 mg/mL to 3 mg/mL; the reaction is carried out for 5 min to 15 min; the NY solution has a concentration of 2 μM, and the NY is any one selected from the group consisting of thiacloprid, omethoate, abamectin, pyridaben, folpet, captan, alachlor, and atrazine; the chromogenic reagent is a mixed solution of TMB, H₂O₂, and NaAc-HAC with a pH of 4.0; in the chromogenic reagent, the TMB, the H₂O₂, and the NaAc-HAC with a pH of 4.0 are mixed in a ratio of (0.4 mL-0.8 mL):(0.4 mL-0.8 mL):(0.1 mL-0.8 mL), and a concentration ratio of the TMB to the H₂O₂ is 1:20 to 5:1; and the volume V4, the volume V5, and the volume V6 are in a ratio of 1:1:1, and are each 10 μL to 20 μL.

Further, in the step 2.1, a calculation equation of the gray value is as follows:

$$Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}}$$

where R refers to a red value extracted from an image, G refers to a green value extracted from an image, B refers to a blue value extracted from an image, and Gray refers to a gray value.

Further, in the step 2.2, the NY solutions with different concentrations are in a concentration range of 0 μM to 20 μM; and the NY is any one selected from the group consisting of thiacloprid, omethoate, abamectin, pyridaben, folpet, captan, alachlor, and atrazine.

Further, in the step 2.2, the first reaction and the second reaction are each carried out for 5 min to 10 min.

Further, in the step 2.2, the volume V7, the volume V8, and the volume V9 are in a ratio of 1:1:1, and are each 10 μL to 20 μL.

Further, in the step 2.4, the volume V10, the volume V11, and the volume V12 are in a ratio of 1:1:1, and are each 10 μL to 20 μL.

Further, in the step 2.4, the reaction is carried out for 5 min to 10 min.

Further, in the step 2.4, the sample is pretreated as follows: crushing the sample first, conducting extraction with acetonitrile and rotary evaporation, and then dissolving a residue in water to obtain the test sample solution.

Further, in the step 2.5, the reaction is carried out for 5 min to 10 min.

The present disclosure also provides use of a standard colorimetric card prepared by the above method in the detection of a pesticide residue in an actual sample.

Compared with the Prior Art, the Present Disclosure has the Following Beneficial Effects.

(1) The imprinted MOF enzyme-mimic probe of the present disclosure has a pesticide-specific recognition site, which effectively overcomes the interference of other components in a complex matrix sample.

(2) The colorimetric test paper of the present disclosure can be prepared by directly adding an imprinted MOF enzyme-mimic probe solution dropwise to an ordinary filter paper without the technical assistance such as specific printing and etching, which has advantages such as low cost, simple operations, and strong practicability.

(3) In the present disclosure, a low-coat filter paper is used as a substrate, an MOF is used to catalyze the oxidation of the substrate for color development, and a color signal is acquired through a camera function of a smartphone, which does not require special instruments such as a fluorescence excitation light source and a signal acquisition device and thus can achieve the convenient, on-site, and visual colorimetric detection of a pesticide residue in a field environment where resources are scarce.

(4) The color development of the MOF-catalyzed substrate designed in the present disclosure can be realized within 5 min, and the color developed can be maintained for 20 min, which can meet the requirements of rapid color development and stable color acquisition.

(5) In the present disclosure, the colorimetric test paper is divided into a quality control zone, a standard zone, and a detection zone. The quality control zone is configured to screen the optimal colorimetric analysis parameters on site, thereby effectively overcoming the measurement errors caused by differences of analysis environments. A color of the detection zone can be compared with a color of the standard zone to preliminarily determine a concentration range of a pesticide residue in a test sample, and then a too-high or too-low concentration is adjusted to meet the needs of accurate determination, which improves the detection accuracy to a large extent. An RGB value can be extracted by a smartphone and a Gray value thereof can be calculated to overcome the influence of a color of a test sample itself on a colorimetric signal, which further improves the reliability of sensor analysis.

### BRIEF DESCRIPTION OF THE DRAWINGS

FIG. 1 shows a scanning electron microscopy (SEM) image of an imprinted MOF enzyme-mimic probe.

FIG. 2 shows a transmission electron microscopy (TEM) image of an imprinted MOF enzyme-mimic probe.

FIG. 3 is a schematic structural diagram of a colorimetric sensing test strip.

FIG. 4 shows a colorimetric sensing test strip prepared in an embodiment.

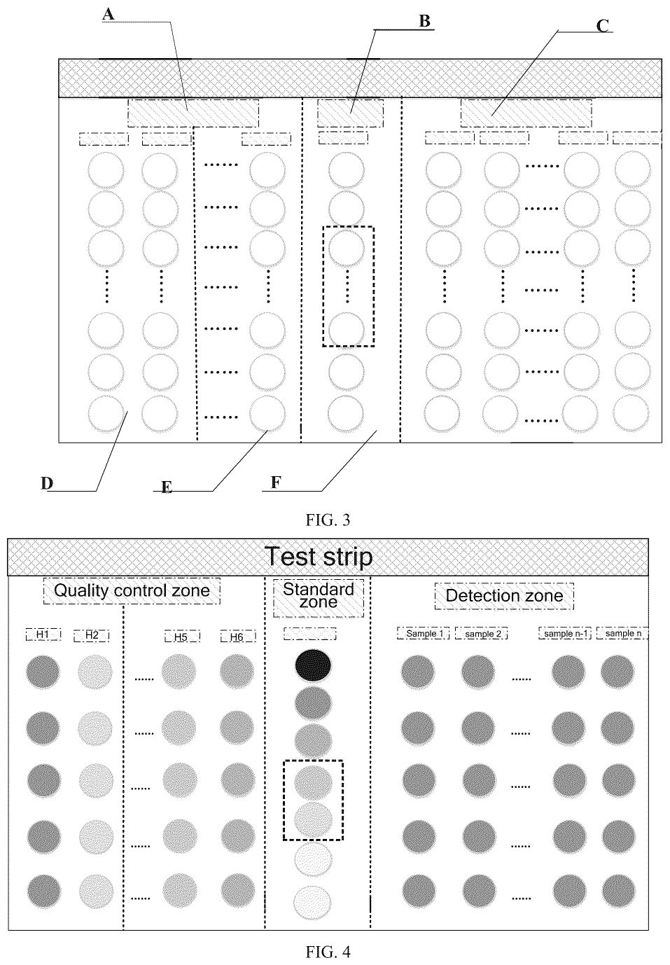

In FIG. 3, A represents a quality control zone; B represents a standard zone; C represents a detection zone; D represents a quality control subzone 1; E represents a quality control subzone 2; and F represents a standard zone.

### DETAILED DESCRIPTION OF THE EMBODIMENTS

The present disclosure will be further described below in conjunction with the accompanying drawings and specific examples, but the protection scope of the present disclosure is not limited thereto.

### Example 1

With the detection of thiacloprid as an example, a colorimetric test paper for rapid detection of thiacloprid was prepared.

Step 1: Preparation of a colorimetric test paper, mainly including: synthesis of an imprinted MOF enzyme-mimic probe, preparation of a blank filter paper, and construction of a colorimetric sensing interface.

Step 1.1: Preparation of an imprinted MOF enzyme-mimic probe (an imprinted MOF enzyme-mimic probe capable of specifically recognizing thiacloprid): 500 mg of an MOF and 20 μL of APTES were dissolved in 2 mL of 10% ammonia water to obtain a mixed solution, then 10 mg of thiacloprid was added to the mixed solution, and the resulting mixture was stirred for 5 min; and 5 mL of TEOS was added, the resulting mixture was stirred and centrifuged, and the resulting precipitate was washed and dried to obtain the imprinted MOF enzyme-mimic probe.

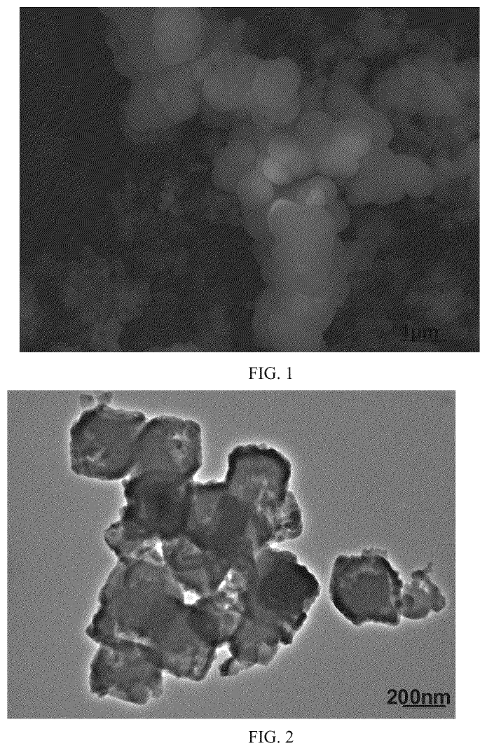

FIG. 1 shows an SEM image of an imprinted MOF enzyme-mimic probe. It can be seen from the figure that the imprinted MOF enzyme-mimic probe prepared by the present disclosure has a regular morphology, and a molecularly imprinted polymer (MIP) layer is formed on a surface of the MOF. FIG. 2 shows a TEM image of an imprinted MOF enzyme-mimic probe. It can be seen from the figure that the polymer layer is evenly distributed on the surface of the MOF with a thickness of about 28 nm.

Step 1.2: Preparation of a blank filter paper: A cheap ordinary filter paper was divided into a zone A, a zone B, and a zone C, where the zone A was a quality control zone, the zone B was a standard zone, and the zone C was a detection zone; and the zone A, the zone B, and the zone C were in an area ratio of 2:1:2.

Step 1.3: Construction of a colorimetric sensing interface: 10 μL of an ultrasonically-homogenized imprinted MOF enzyme-mimic probe solution was directly added dropwise to the quality control zone on the blank filter paper, and the quality control zone was allowed to be dried, which were simple operations without procedures such as printing. The quality control zone was equally divided into a quality control subzone 1 and a quality control subzone 2. The quality control subzone 1 was equally divided into 3 zones from left to right (namely, 3 columns) that were denoted as H₁, H₂, and H₃, respectively; and the quality control subzone 2 was also equally divided into 3 zones from left to right (namely, 3 columns) that were denoted as I₁, I₂, I₃, respectively.

FIG. 3 is a schematic structural diagram of a colorimetric sensing test strip, where A represents a quality control zone; B represents a standard zone; C represents a detection zone; the quality control zone A is divided into a quality control subzone 1 and a quality control subzone 2; D represents an optimized imprinted MOF enzyme-mimic probe solution concentration zone in the quality control subzone 1; E represents an optimized chromogenic reagent concentration zone in the quality control subzone 2; F represents a chromogenic zone corresponding to the determination of a standard pesticide sample in the standard zone; the black dotted box in the standard zone is provided to preliminarily determine a concentration range of a pesticide residue, such that a too-high or too-low concentration can be adjusted; the zone A, the zone B, and the zone C are in an area ratio of 2:1:2; and the detection zone can be used for the simultaneous on-line detection of multiple samples.

Step 2.1: Establishment of the quality control zone:

Step 2.1.1: Determination of the optimal concentration of an imprinted MOF enzyme-mimic probe solution

Imprinted MOF enzyme-mimic probe solutions with concentrations of 1 mg/mL, 2 mg/mL, and 3 mg/mL were taken, numbered as 1, 2, and 3, and added each in a volume of 10 μL dropwise to the zones H₁, H₂, and H₃ of the quality control subzone 1, and the zones were allowed to be dried; then thiacloprid was added to water to obtain a thiacloprid standard solution; 10 μL of a 2 μM thiacloprid standard solution was added dropwise to each of H₁, H₂, and Ha of the quality control subzone 1, a reaction was carried out for 10 min, and then 10 μL of a chromogenic reagent (including TMB, hydrogen peroxide (H₂O₂), and NaAc-HAC with a pH of 4.0) was then added dropwise; an image of each zone was acquired, and a gray value was further calculated according to a corresponding RGB value; and a concentration of the imprinted MOF enzyme-mimic probe solution corresponding to a zone with the largest gray value was determined as the optimal concentration of the imprinted MOF enzyme-mimic probe solution. In this case, the optimal concentration of the imprinted MOF enzyme-mimic probe solution was 3 mg/mL.

Step 2.1.2: Determination of an optimal chromogenic reagent concentration:

10 μL of a 3 mg/mL imprinted MOF enzyme-mimic probe solution was added dropwise to each of I₁, I₂, and I₃ of the quality control subzone 2, and the zones were allowed to be dried; 10 μL of a 2 μM thiacloprid standard solution was added dropwise to each of I₁, I₂, and I₃ of the quality control subzone 2, a reaction was carried out for 10 min, and chromogenic reagents with different concentrations were added each in a volume of 10 μL dropwise (with a concentration ratio of TMB to H₂O₂ as a basis for determination, the following specific 3 groups were set: 1:20, 1:2, and 5:1); an image of each zone was acquired, and a gray value was further calculated according to a corresponding RGB value; and a chromogenic reagent concentration corresponding to a zone with the largest gray value was determined as the optimal chromogenic reagent concentration, where a chromogenic reagent with the optimal concentration was a mixed solution including 0.4 mL of TMB (0.05 M), 0.1 mL of H₂O₂ (10 M), and 0.5 mL of NaAc-HAC with a pH of 4.0 (0.1 M).

Step 2.2: Establishment of the standard zone:

According to the optimization results in the step 2.1, the standard zone was divided into 6 zones from top to bottom that were denoted as E₁, E₂, E₃, E₄, E₅, and E₆, respectively; with the optimal concentration of the imprinted MOF enzyme-mimic probe solution determined in the step 2.1.1, 10 μL of a 3 mg/mL imprinted MOF enzyme-mimic probe solution was added dropwise to a surface of each of E₁, E₂, E₃, E₄, E₅, and E₆ of the standard zone, and the zones were allowed to be dried; thiacloprid standard solutions with concentrations of 0 μM, 0.3 μM, 0.5 μM, 1.2 μM, 2 μM, and 8 μM were prepared, denoted as C₁, C₂, C₃, C₄, C₅, and C₆, and added each in a volume of 10 μL dropwise to E₁, E₂, E₃, E₄, E₅, and E₆, respectively, and a reaction was carried out for 10 min; and then with the optimal chromogenic reagent concentration obtained in the step 2.1.2 (the chromogenic reagent was a mixed solution including 0.4 mL of TMB (0.05 M), 0.1 mL of H₂O₂ (10 M), and 0.5 mL of NaAc-HAC with a pH of 4.0 (0.1 M)), 10 μL of the chromogenic reagent was added dropwise to each of E₁, E₂, E₃, E₄, E₅, and E₆, and a reaction was carried out for 10 min to establish a standard colorimetric card for the standard zone. A color of the standard colorimetric card for the standard zone remained unchanged for 20 min or more, which reserved enough time for the subsequent preliminary determination of a concentration of a pesticide residue in the detection zone.

Step 2.3: Chromogenic images of different pesticide concentrations corresponding to the standard colorimetric card for the standard zone prepared in the step 2.2 were acquired through a camera function of a smartphone, and then the RGB values for different thiacloprid standard solution concentrations were analyzed through mobile phone software. RGB values for 0 μM, 0.3 μM, 0.5 μM, 1.2 μM, 2 μM, and 8 μM were {169, 192, 187}, {163, 181, 177}, {154, 173, 169}, {156, 171, 166}, {152, 168, 163}, and {149, 166, 159}, respectively.

$$According to equation ( 1 ) Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}} ,$$

corresponding Gray values G₁=186.06, G₂=167.91, G₃=163.98, G₄=161.83, G₅=147.51, and G₆=138.11 were obtained through calculation, a median M for the Gray values of the quality control zone was calculated as follows: (G₃+G₄)/2=162.9, and based on this, the optimal gray value for sample analysis was determined, that is, a discriminant value P was 162.9×(1±10%). In addition, according to a correlation between the Gray values of the standard zone and the thiacloprid standard solution concentrations, a colorimetric analysis mathematical model was established to be Y=−5.87 m+169.4 (m was in a range of 0.3 μM to 2 μM), with a detection limit of 0.134 μM.

### Example 2: Detection of Thiacloprid in Actual Samples

Step 1.1: Green tea, dark tea, soil, apple, Romaine lettuce, and Indian lettuce were denoted as a test sample 1, a test sample 2, a test sample 3, a test sample 4, a test sample 5, and a test sample 6, pretreated, and subjected to extraction with acetonitrile and rotary evaporation, and resulting residues were each dissolved in water to obtain corresponding test sample solutions.

Step 1.2: Establishment of the detection zone:

The detection zone was evenly divided into 6 sample zones from left to right (namely, 6 columns) that were denoted as a sample zone 1, a sample zone 2, a sample zone 3, a sample zone 4, a sample zone 5, and a sample zone 6; the sample zone 1 was divided into 5 subzones that were denoted as 1₁, 1₂, 1₃, 1₄, and 1₅, respectively, the sample zone 2 was divided into 5 subzones that were denoted as 2₁, 2₂, 2₃, 2₄, and 2₅, respectively, the sample zone 3 was divided into 5 subzones that were denoted as 3₁, 3₂, 3₃, 3₄, and 3₅, respectively, the sample zone 4 was divided into 5 subzones that were denoted as 4₁, 4₂, 4₃, 4₄, and 4₅, respectively, the sample zone 5 was divided into 5 subzones that were denoted as 5₁, 5₂, 5₃, 5₄, and 5₅, respectively, and the sample zone 6 was divided into 5 subzones that were denoted as 6₁, 6₂, 6₃, 6₄, and 6₅, respectively; and with the optimal imprinted MOF probe concentration 3 mg/mL obtained in the step 2.1.1, 10 μL of a 3 mg/mL imprinted MOF enzyme-mimic probe solution was added dropwise to each of 5 subzones of each of the 6 sample zones of the detection zone, and the subzones were allowed to be dried, such that the detection zone was established.

Step 1.3: 10 μL of a test sample solution 1 obtained in the step 1.1 was added dropwise to a surface of each of the detection zones 1₁, 1₂, 1₃, 1₄, and 1₅ of the test strip in the step 1.2, 10 μL of a test sample solution 2 was added dropwise to a surface of each of the detection zones 2₁, 2₂, 2₃, 2₄, and 2₅ of the test strip in the step 1.2, 10 μL of a test sample solution 3 was added dropwise to a surface of each of the detection zones 3₁, 3₂, 3₃, 3₄, and 3₅ of the test strip in the step 1.2, 10 μL of a test sample solution 4 was added dropwise to a surface of each of the detection zones 4₁, 4₂, 4₃, 4₄, and 4₅ of the test strip in the step 1.2, 10 μL of a test sample solution 5 was added dropwise to a surface of each of the detection zones 5₁, 5₂, 5₃, 5₄, and 5₅ of the test strip in the step 1.2, and 10 μL of a test sample solution 6 was added dropwise to a surface of each of the detection zones 6₁, 6₂, 6₃, 6₄, and 6₅ of the test strip in the step 1.2; and a reaction was carried out for 10 min, and 10 μL of the chromogenic reagent with the optimal concentration was added to each of the above zones (the chromogenic reagent with the optimal concentration was a mixed solution of 0.5 mL of TMB, 0.4 mL of H₂O₂, and 0.1 mL of NaAc-HAC with a pH of 4.0, in which a concentration ratio of TMB to H₂O₂ was 1:20).

Step 1.4: After the chromogenic reagent was added dropwise in the step 1.3, a reaction was carried out for 5 min, then RGB values of different zones were acquired through photographing with a mobile phone, and corresponding Gray values are calculated. If a gray value was not within 162.9×(1±10%), the original sample needed to be adjusted, then the detection zone establishment in the step 2.4 was repeated, and then an RGB value was acquired through photographing with a mobile phone and a Gray value was calculated.

A color for sample 1 was compared with the standard zone, and a concentration range of a pesticide residue was preliminarily determined to be 0.5 μM to 1.2 μM; a picture was taken, and then a gray value of a chromogenic zone of sample 1 was calculated to be 163.5, which was within 162.9×(1±10%); and there was no need to adjust the pesticide residue concentration in sample 1, and thus the gray value 163.5 of sample 1 was directly substituted into the colorimetric analysis mathematical model to obtain a pesticide residue concentration of 0.4 μM.

A color for sample 2 was compared with the standard zone, and a concentration range of a pesticide residue was preliminarily determined to be 2 μM to 4 μM; a picture was taken, and then a gray value of a chromogenic zone of sample 2 was calculated to be 120.8, which was not within 162.9×(1±10%); it was preliminarily determined that the pesticide residue concentration was too high; and in order to ensure the accuracy, sample 2 was diluted 0.5 times, then a gray value thereof was further calculated to be 152.4 and substituted into the colorimetric analysis mathematical model to obtain a pesticide residue concentration of 1.3 μM, and the pesticide residue concentration in sample 2 was calculated as follows: 1.3 μM×2=2.6 μM.

A color for sample 3 was compared with the standard zone, and a concentration range of a pesticide residue was preliminarily determined to be 0 μM to 0.5 μM; a picture was taken, and then a gray value of a chromogenic zone of sample 3 was calculated to be 190.5, which was not within 162.9×(1±10%); it was preliminarily determined that the pesticide residue concentration was too low; and in order to ensure the accuracy, sample 3 was concentrated 5 times, then a gray value thereof was further calculated to be 145.8 and substituted into the colorimetric analysis mathematical model to obtain a pesticide residue concentration of 1.2 μM, and the pesticide residue concentration in sample 3 was calculated as follows: 1.2 μM+5=0.24 μM.

A color for sample 4 was compared with the standard zone, and a concentration range of a pesticide residue was preliminarily determined to be 0.5 μM to 1.2 μM; a picture was taken, and then a gray value of a chromogenic zone of sample 4 was calculated to be 166.5, which was within 162.9×(1±10%); and there was no need to adjust the pesticide residue concentration in sample 4, and thus the gray value 166.5 of sample 4 was directly substituted into the colorimetric analysis mathematical model to obtain a pesticide residue concentration of 0.5 μM.

A color for sample 5 was compared with the standard zone, and a concentration range of a pesticide residue was preliminarily determined to be 0.5 μM to 1.2 μM; a picture was taken, and then a gray value of a chromogenic zone of sample 5 was calculated to be 175.5, which was within 162.9×(1±10%); and there was no need to adjust the pesticide residue concentration in sample 5, and thus the gray value 175.5 of sample 5 was directly substituted into the colorimetric analysis mathematical model to obtain a pesticide residue concentration of 0.63 μM.

A color for sample 6 was compared with the standard zone, and a concentration range of a pesticide residue was preliminarily determined to be 0 μM to 0.5 μM; a picture was taken, and then a gray value of a chromogenic zone of sample 6 was calculated to be 150, which was within 162.9×(1±10%); and there was no need to adjust the pesticide residue concentration in sample 6, and thus the gray value 150 of sample 6 was directly substituted into the colorimetric analysis mathematical model to obtain a pesticide residue concentration of 0.32 μM.

FIG. 4 is a schematic diagram illustrating the detection of thiacloprid residues in different test samples, where a zone in the black dotted box shows the color development corresponding to a thiacloprid standard in the standard zone, which is provided to preliminarily determine a median for pesticide residues in test samples.

In order to further verify the accuracy and sensitivity of the constructed test strip, the colorimetric sensing system of the present disclosure was compared with standard HPLC. Results were shown in Table 1. A relative standard deviation (RSD) of the detection results of the present disclosure is 3.4% to 5.8%, which is within an acceptable range; and the RSD value of the test strip detection method is slightly smaller than an RSD value of the standard method HPLC, indicating that the test strip detection method of the present disclosure can lead to relatively-stable results with excellent reproducibility.

TABLE 1
Comparison between the colorimetric
array and the HPLC detection method

| Sample | Colorimetric | RSD | HPLC | RSD |
|---|---|---|---|---|
| No. | array (μM) | (%) | (μM) | (%) |
| 1 | 0.40 | 4.3 | 0.38 | 5.6 |
| 2 | 2.62 | 5.8 | 2.70 | 6.9 |
| 3 | 0.24 | 3.4 | 0.19 | 4.8 |
| 4 | 0.52 | 4.2 | 0.49 | 6.5 |
| 5 | 0.63 | 5.1 | 0.68 | 4.3 |
| 6 | 0.32 | 3.9 | 0.40 | 5.8 |

The colorimetric array method has high sensitivity, excellent stability, and prominent specificity due to the following reasons: (1) The imprinted MOF enzyme-mimic probe of the present disclosure does not require the use of biomolecules such as antibodies and aptamers, and can be used for the specific recognition and in-situ catalysis of a target in a specific environment, thereby improving the detection sensitivity and the anti-interference ability of a sensing system. (2) The colorimetric analysis method of the present disclosure involves simple operation, and does not require special instruments such as a fluorescence excitation light source and a signal acquisition device. (3) The test strip of the present disclosure is divided into a quality control zone, a detection zone, and a standard zone, where the quality control zone is configured to select the optimal colorimetric parameters for on-site analysis, thereby effectively overcoming experimental errors caused by environmental differences; the standard zone is configured to establish a colorimetric analysis mathematical model and preliminarily determine a pesticide residue content in an actual sample, such that a too-high or too-low pesticide residue concentration can be adjusted to ensure the analysis accuracy; and an RGB value of an image can be acquired by a mobile phone, and a Gray value is calculated as a colorimetric signal, which effectively avoids the interference of a color of a sample itself on a detection result.

In summary, in the present disclosure, a molecularly imprinted MOF is used as a colorimetric probe to specifically recognize different pesticide residues and catalyze the oxidization of a substrate to allow a chromogenic reaction; and a colorimetric test strip is constructed with a low-cost filter paper and divided into a quality control zone, a standard zone, and a detection zone, where the quality control zone effectively overcomes the shortcoming that the existing test strip is prone to environmental interference; and the detection zone and the standard zone can be compared to preliminarily determine a pesticide residue content in an actual sample, such that a too-high or too-low pesticide residue concentration can be adjusted to ensure the analysis accuracy. In addition, an RGB color signal of a chromogenic system can be stably captured by a smartphone, and according to a correlation between the Gray values and the pesticide residue concentrations, the highly-sensitive and specific colorimetric analysis of a trace pesticide residue is realized. The colorimetric sensing analysis method has advantages such as high efficiency, high sensitivity, and prominent reliability, and provides a new technical support for field monitoring of a pesticide residue in a complex matrix such as an environment and an agricultural product.

The series of detailed description listed above are only specific illustration of feasible examples of the present disclosure, rather than limitation of the claimed scope of the present disclosure. All equivalent examples or changes made without departing from the technical spirit of the present disclosure should be included in the claimed scope of the present disclosure.

What is claimed is:

1. A method for highly-sensitive and rapid detection of a pesticide residue based on an imprinted metal-organic framework (MOF) probe, comprising the following steps:
step 1.1: dissolving an MOF and aminopropyltriethoxysilane in ammonia water to obtain a mixed solution; selecting a pesticide standard and denoting the pesticide standard as NY; adding the pesticide standard to the mixed solution, subjecting a resulting mixture to a first stirring, adding tetraethylorthosilicate, and subjecting a resulting mixture to a second stirring; and then centrifuging, washing, and drying to obtain an imprinted MOF enzyme-mimic probe;
step 1.2: taking an ordinary filter paper, and dividing the ordinary filter paper into a first zone, a second zone, and a third zone, wherein the first zone is a quality control zone, the second zone is a standard zone, and the third zone is a detection zone;
step 1.3: dividing the quality control zone into a first quality control subzone and a second quality control subzone; dividing the first quality control subzone into n zones from left to right that are denoted as H₁, H₂, H₃ . . . Hₙ₋₁, and Hₙ, respectively; and dividing the second quality control subzone into m zones from left to right that are denoted as I₁, I₂, I₃ . . . Iₘ₋₁, and Iₘ, respectively, wherein n and m are each an integer greater than 1;
step 2.1: establishment of the quality control zone:
step 2.1.1: determination of an optimal concentration of an imprinted MOF enzyme-mimic probe solution:
adding the imprinted MOF enzyme-mimic probe prepared in the step 1.1 to ethanol to obtain imprinted MOF enzyme-mimic probe solutions with different concentrations that are denoted as 1, 2 . . . n−1, and n, respectively, adding the imprinted MOF enzyme-mimic probe solutions 1, 2 . . . n−1, and n in a volume V1 dropwise to zones H₁, H₂, H₃ . . . Hₙ₋₁, and Hₙ of the first quality control subzone, respectively, and allowing the zones to be dried; then dissolving the NY in the step 1.1 into water to obtain an NY solution; adding the NY solution in a volume V2 dropwise to each of H₁, H₂, H₃ . . . Hₙ₋₁, and Hₙ of the first quality control subzone, and allowing a first reaction to occur for a period of time; adding a chromogenic reagent in a volume V3 dropwise to each of H₁, H₂, H₃ . . . Hₙ₋₁, and Hₙ of the first quality control subzone; observing color changes of the zones H₁, H₂, H₃ . . . Hₙ₋₁, and Hₙ of the first quality control subzone, acquiring an image and a corresponding RGB value of each of the zones, and further calculating a gray value; and determining a concentration of an imprinted MOF enzyme-mimic probe solution corresponding to a zone with the largest gray value as the optimal concentration of the imprinted MOF enzyme-mimic probe solution, wherein the chromogenic reagent comprises 3,3′,5,5′-tetramethylbenzidine (TMB), hydrogen peroxide (H₂O₂), and NaAc-HAC with a pH of 4.0;
step 2.1.2: determination of an optimal chromogenic reagent concentration:
after the determination of the optimal concentration of the imprinted MOF enzyme-mimic probe solution in the step 2.1.1, adding the imprinted MOF enzyme-mimic probe solution with the optimal concentration in a volume V4 dropwise to zones I₁, I₂, I₃ . . . Iₘ₋₁, and Iₘ of the second quality control subzone, and allowing the zones to be dried; adding the NY solution in the step 2.1.1 in a volume V5 dropwise to each of I₁, I₂, I₃ . . . Iₘ₋₁, and Iₘ of the second quality control subzone, and allowing a second reaction to occur for a period of time; adding the chromogenic reagent with different concentrations in a volume V6 dropwise to I₁, I₂, I₃ . . . Iₘ₋₁, and Iₘ of the second quality control subzone, respectively; observing color changes of I₁, I₂, I₃ . . . Iₘ₋₁, and Iₘ of the second quality control subzone, acquiring an image and a corresponding RGB value of each of the zones, and further calculating a gray value; and determining a chromogenic reagent concentration corresponding to a zone with the largest gray value as the optimal chromogenic reagent concentration, wherein the chromogenic reagent comprises TMB, H₂O₂, and NaAc-HAC with a pH of 4.0;
step 2.2: establishment of the standard zone:
dividing the standard zone into n zones from top to bottom that are denoted as E₁, E₂, E₃ . . . Eₙ₋₁, and En, respectively; after the determination of the optimal concentration of the imprinted MOF enzyme-mimic probe solution in the step 2.1.1, adding the imprinted MOF enzyme-mimic probe solution with the optimal concentration in a volume V7 dropwise to a surface of each of E₁, E₂, E₃ . . . Eₙ₋₁, and Eₙ of the standard zone, and allowing the surfaces to be dried; preparing NY solutions with different concentrations, and denoting the NY solutions with different concentrations as C₁, C₂ . . . Cₙ₋₁, and Cₙ; adding the NY solutions with different concentrations in a volume V8 dropwise to E₁, E₂, E₃ . . . Eₙ₋₁, and Eₙ of the standard zone, respectively, and allowing a third reaction; and with the optimal chromogenic reagent concentration determined in the step 2.1.2, adding the chromogenic reagent in a volume V9 to each of E₁, E₂, E₃ . . . Eₙ₋₁, and Eₙ of the standard zone, and allowing a fourth reaction, so as to establish a standard colorimetric card for the standard zone, wherein a color of the standard colorimetric card for the standard zone remains unchanged for 20 min or more;
step 2.3: acquiring chromogenic images of the NY solutions with different concentrations corresponding to the standard colorimetric card for the standard zone in the step 2.2, and analyzing RGB values of the NY solutions with different concentrations; calculating corresponding Gray values according to equation (1), and denoting the Gray values as G₁, G₂, G₃ . . . Gₙ₋₁, and Gₙ, respectively,
$$Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}} , \qquad ( 1 )$$
wherein R refers to a red value extracted from an image, G refers to a green value extracted from an image, B refers to a blue value extracted from an image, and Gray refers to a gray value;
establishing a colorimetric analysis mathematical model as follows based on the Gray values of the standard zone calculated by equation (1) and corresponding NY solution concentrations m: Y=k*m+b, wherein b and k are each a constant, and m is an NY solution concentration, in a unit of μM; and
denoting a discriminant value for the NY solution in the standard zone as P with a value range of M*(1−10%) to M*(1+10%), wherein M is a median for gray values corresponding to different NY solution concentrations in the standard zone;
step 2.4: establishment of the detection zone:
equally dividing the detection zone into N zones from left to right that are denoted as a sample zone 1, a sample zone 2, a sample zone 3 . . . a sample zone n−1, and a sample zone n; dividing the sample zone 1 into zones 1₁, 1₂, 1₃ . . . lᵢ, dividing the sample zone 2 into zones 2₁, 2₂, 2₃ . . . 2ᵢ, dividing the sample zone 3 into zones 3₁, 3₂, 3₃ . . . 3ᵢ, . . . dividing the sample zone n−1 into zones n−1₁, n−1₂, n−1₃ . . . n−1ᵢ, and dividing the sample zone n into zones n₁, n₂, n₃ . . . nᵢ;
pretreating n test samples to obtain test sample solutions that are numbered as a test sample solution 1, a test sample solution 2 . . . a test sample solution N, respectively; adding the imprinted MOF enzyme-mimic probe solution with the optimal concentration obtained according to the step 2.1.1 in a volume V10 dropwise to each of the N zones of the detection zone, and allowing the zones to be dried; and adding the test sample solution 1 in a volume V11 dropwise to each of 1₁, 1₂, 1₃ . . . 1ᵢ, adding the test sample solution 2 in a volume V11 dropwise to each of 2₁, 2₂, 2₃ . . . adding the test sample solution n−1 in a volume V11 dropwise to each of n−1₁, n−1₂, n−1₃ . . . n−1ᵢ, and adding the test sample solution n in a volume V11 dropwise to each of n₁, n₂, n₃ . . . nᵢ, and allowing a fifth reaction to occur for a period of time, wherein i and n are each an integer greater than 1;
step 2.5: adding the chromogenic reagent with the optimal concentration obtained in the step 2.1.2 in a volume V12 dropwise to the N zones of the detection zone obtained in the step 2.4, and allowing a sixth reaction to occur for a period of time; comparing colors of the N zones of the detection zone with colors of the standard colorimetric card for the standard zone established in the step 2.2, and preliminarily determining a concentration range of the pesticide residue in the test sample; and calculating a Gray value of the pesticide residue in the test sample according to equation (1), and when the Gray value is not in a value range of the discriminant value P, adjusting the concentration of the pesticide residue in the test sample,
wherein if the Gray value of the concentration of the pesticide residue in the test sample is greater than M*(1+10%), the test sample needs to be diluted until the Gray value is within the value range of the discriminant value P, and a dilution ratio is recorded; and
if the Gray value of the concentration of the pesticide residue in the test sample is smaller than the median M*(1−10%), the test sample needs to be concentrated until the Gray value is within the value range of the discriminant value P, and a concentration ratio is recorded;
after adjustment of the concentration of the pesticide residue, repeating the steps 2.4 and 2.5, calculating a Gray value G₀ of a chromogenic image according to equation (1), and comparing the Gray value with the discriminant value P; and if the G₀ value is within the value range of the discriminant value P, calculating a content of the pesticide residue in the test sample according to the colorimetric analysis mathematical model: Y₀=(G₀−b)/k, wherein M is a median for gray values corresponding to different NY solution concentrations in the standard zone, b and k are each a constant, and Y₀ is an adjusted pesticide concentration in the test sample.

2. The method for highly-sensitive and rapid detection of the pesticide residue based on the imprinted MOF probe according to claim 1, wherein, in the step 1.1, the MOF, the aminopropyltriethoxysilane, the ammonia water, the pesticide standard, and the tetraethylorthosilicate are used in a ratio of (400-700) mg:(10-30) μL:(2-10) mL:(10-20) mg:(5-15) mL; the ammonia water has a volume fraction of 5% to 15%; the first stirring and the second stirring are each performed for 5 min to 15 min; and
the pesticide standard comprises an insecticide, a miticide, a bactericide, and an herbicide, and is specifically any one selected from the group consisting of thiacloprid, omethoate, abamectin, pyridaben, folpet, captan, alachlor, and atrazine.

3. The method for highly-sensitive and rapid detection of the pesticide residue based on the imprinted MOF probe according to claim 1, wherein, in the step 1.2, the first zone, the second zone, and the third zone are in an area ratio of 2:1:2.

4. The method for highly-sensitive and rapid detection of the pesticide residue based on the imprinted MOF probe according to claim 1, wherein, in the step 2.1.1, the NY solution has a concentration of 2 μM, and the NY is any one selected from the group consisting of thiacloprid, omethoate, abamectin, pyridaben, folpet, captan, alachlor, and atrazine; the first reaction is carried out for 5 min to 10 min; the imprinted MOF enzyme-mimic probe solution has a concentration of 1 mg/mL to 3 mg/mL; the chromogenic reagent is a mixed solution of TMB, H₂O₂, and NaAc-HAC with a pH of 4.0; in the chromogenic reagent, the TMB, the H₂O₂, and the NaAc-HAC with a pH of 4.0 are mixed in a ratio of (0.4 mL-0.8 mL):(0.4 mL-0.8 mL):(0.1 mL-0.8 mL), and a concentration ratio of the TMB to the H₂O₂ is 1:20 to 5:1; and
the volume V1, the volume V2, and the volume V3 are in a ratio of 1:1:1, and are each 10 μL to 20 μL.

5. The method for highly-sensitive and rapid detection of the pesticide residue based on the imprinted MOF probe according to claim 1, wherein, in the step 2.1.2, the imprinted MOF enzyme-mimic probe solution has a concentration of 1 mg/ml to 3 mg/mL; the second reaction is carried out for 5 min to 15 min; the NY solution has a concentration of 2 μM, and the NY is any one selected from the group consisting of thiacloprid, omethoate, abamectin, pyridaben, folpet, captan, alachlor, and atrazine; the chromogenic reagent is a mixed solution of TMB, H₂O₂, and NaAc-HAC with a pH of 4.0; in the chromogenic reagent, the TMB, the H₂O₂, and the NaAc-HAC with a pH of 4.0 are mixed in a ratio of (0.4 mL-0.8 mL):(0.4 mL-0.8 mL):(0.1 mL-0.8 mL), and a concentration ratio of the TMB to the H₂O₂ is 1:20 to 5:1; and
the volume V4, the volume V5, and the volume V6 are in a ratio of 1:1:1, and are each 10 μL to 20 μL.

6. The method for highly-sensitive and rapid detection of the pesticide residue based on the imprinted MOF probe according to claim 1, wherein, in the step 2.1, a calculation equation of the gray value is as follows:
$$Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}} ,$$
wherein R refers to a red value extracted from an image, G refers to a green value extracted from an image, B refers to a blue value extracted from an image, and Gray refers to a gray value.

7. The method for highly-sensitive and rapid detection of the pesticide residue based on the imprinted MOF probe according to claim 1, wherein, in the step 2.2, the NY solutions with different concentrations are in a concentration range of 0 μM to 20 μM; the NY is any one selected from the group consisting of thiacloprid, omethoate, abamectin, pyridaben, folpet, captan, alachlor, and atrazine;
the third reaction and the fourth reaction are each carried out for 5 min to 10 min; and
the volume V7, the volume V8, and the volume V9 are in a ratio of 1:1:1, and are each 10 μL to 20 μL.

8. The method for highly-sensitive and rapid detection of the pesticide residue based on the imprinted MOF probe according to claim 1, wherein, in the step 2.4, the volume V10, the volume V11, and the volume V12 are in a ratio of 1:1:1, and are each 10 μL to 20 μL; the fifth reaction is carried out for 5 min to 10 min; and a sample is pretreated as follows: crushing the sample first, conducting extraction with acetonitrile and rotary evaporation, and then dissolving a residue in water to obtain the test sample solution.

9. The method for highly-sensitive and rapid detection of the pesticide residue based on the imprinted MOF probe according to claim 1, wherein, in the step 2.5, the sixth reaction is carried out for 5 min to 10 min.

10. Use of the standard colorimetric card prepared by the method according to claim 1 in the rapid detection of a pesticide residue.

11. The use according to claim 10, wherein, in the step 1.1, the MOF, the aminopropyltriethoxysilane, the ammonia water, the pesticide standard, and the tetraethylorthosilicate are used in a ratio of (400-700) mg:(10-30) μL:(2-10) mL:(10-20) mg:(5-15) mL; the ammonia water has a volume fraction of 5% to 15%; the first stirring and the second stirring are each performed for 5 min to 15 min; and
the pesticide standard comprises an insecticide, a miticide, a bactericide, and an herbicide, and is specifically any one selected from the group consisting of thiacloprid, omethoate, abamectin, pyridaben, folpet, captan, alachlor, and atrazine.

12. The use according to claim 10, wherein, in the step 1.2, the first zone, the second zone, and the third zone are in an area ratio of 2:1:2.

13. The use according to claim 10, wherein, in the step 2.1.1, the NY solution has a concentration of 2 μM, and the NY is any one selected from the group consisting of thiacloprid, omethoate, abamectin, pyridaben, folpet, captan, alachlor, and atrazine; the first reaction is carried out for 5 min to 10 min; the imprinted MOF enzyme-mimic probe solution has a concentration of 1 mg/mL to 3 mg/mL; the chromogenic reagent is a mixed solution of TMB, H₂O₂, and NaAc-HAC with a pH of 4.0; in the chromogenic reagent, the TMB, the H₂O₂, and the NaAc-HAC with a pH of 4.0 are mixed in a ratio of (0.4 mL-0.8 mL):(0.4 mL-0.8 mL):(0.1 mL-0.8 mL), and a concentration ratio of the TMB to the H₂O₂ is 1:20 to 5:1; and
the volume V1, the volume V2, and the volume V3 are in a ratio of 1:1:1, and are each 10 μL to 20 μL.

14. The use according to claim 10, wherein, in the step 2.1.2, the imprinted MOF enzyme-mimic probe solution has a concentration of 1 mg/mL to 3 mg/mL; the second reaction is carried out for 5 min to 15 min; the NY solution has a concentration of 2 μM, and the NY is any one selected from the group consisting of thiacloprid, omethoate, abamectin, pyridaben, folpet, captan, alachlor, and atrazine; the chromogenic reagent is a mixed solution of TMB, H₂O₂, and NaAc-HAC with a pH of 4.0; in the chromogenic reagent, the TMB, the H₂O₂, and the NaAc-HAC with a pH of 4.0 are mixed in a ratio of (0.4 mL-0.8 mL): (0.4 mL-0.8 mL): (0.1 mL-0.8 mL), and a concentration ratio of the TMB to the H₂O₂ is 1:20 to 5:1; and
the volume V4, the volume V5, and the volume V6 are in a ratio of 1:1:1, and are each 10 μL to 20 μL.

15. The use according to claim 10, wherein, in the step 2.1, a calculation equation of the gray value is as follows:
$$Gray = \sqrt[2.2]{\frac{{R}^{2.2} + {( 1.5 G )}^{2.2} + {( 0.6 B )}^{2.2}}{1 + {1.5}^{2.2} + {0.6}^{2.2}}} ,$$
wherein R refers to a red value extracted from an image, G refers to a green value extracted from an image, B refers to a blue value extracted from an image, and Gray refers to a gray value.

16. The use according to claim 10, wherein, in the step 2.2, the NY solutions with different concentrations are in a concentration range of 0 μM to 20 μM; the NY is any one selected from the group consisting of thiacloprid, omethoate, abamectin, pyridaben, folpet, captan, alachlor, and atrazine;
the third reaction and the fourth reaction are each carried out for 5 min to 10 min; and
the volume V7, the volume V8, and the volume V9 are in a ratio of 1:1:1, and are each 10 μL to 20 μL.

17. The use according to claim 10, wherein, in the step 2.4, the volume V10, the volume V11, and the volume V12 are in a ratio of 1:1:1, and are each 10 μL to 20 μL; the fifth reaction is carried out for 5 min to 10 min; and a sample is pretreated as follows: crushing the sample first, conducting extraction with acetonitrile and rotary evaporation, and then dissolving a residue in water to obtain the test sample solution.

18. The use according to claim 10, wherein, in the step 2.5, the sixth reaction is carried out for 5 min to 10 min.
